(19) United States
(12) Patent Application Publication
(10) Pub. No.: US 2026/0325183 A1
(43) Pub. Date: Oct. 22, 2026

(54) MANUFACTURING METHOD FOR EPIPHYSIS PLATES

(71) Applicant: FUJI OOZX Inc., Shizuoka (JP)

(72) Inventors: Taiga SASAKI, Shizuoka (JP); Koji KUSHIMA, Shizuoka (JP)

(73) Assignee: FUJI OOZX Inc., Shizuoka (JP)

(21) Appl. No.: 19/106,635

(22) PCT Filed: Sep. 7, 2022
(86) PCT No.: PCT/JP2022/033535

§ 371 Date: Feb. 26, 2025

(51) Int. Cl. A61B 17/80; A61B 17/00

(52) U.S. Cl. CPC A61B 17/80; A61B 2017/00526

(57) ABSTRACT

The present invention proposes a manufacturing method for an epiphysis plate that uses forward extrusion forging and can achieve the equiaxed microstructure required by ISO 20160.

A manufacturing method of an epiphysis plate, the method including:
a step of heating a metal material of a round bar; a step of forming a billet having a head portion with a large diameter and a shaft portion with a small diameter integral with the head portion by a forward extrusion device; a step of heating the billet; a step of forming a die forged product close to a shape of the epiphysis plate as a final product by die forging using a die device, and a step of annealing the die forged product.

### TECHNICAL FIELD

The present invention relates to a manufacturing method for an epiphysis plate used to fix a fractured part, and more particularly to a manufacturing method for the epiphysis plate having a complex shape that matches the shape of the epiphysis.

### BACKGROUND OF THE INVENTION

As the aging of society continues to progress in Japan and many other countries around the world, there is an increasing demand for an epiphysis plate to treat fractures caused by falls among the elderly. Here, the epiphysis plate used at the epiphysis part has a complex shape and is thin, so it is manufactured by cutting work of a metal block as a material. In this case, a large amount of cutting waste is generated, which causes a problem of low yield.

Therefore, non-patent literature 1 discloses a high temperature forming process technology without cutting work, with the aim of reducing the generation of cutting waste and improving the yield of metal materials. This technology involves heating a round bar of metal material to a high temperature in a furnace and forming the round bar into the shape of an epiphysis plate by two-stage forging, namely rough forming and finish forming, using a die device.

The high-temperature forming process has significantly improved yield compared to cutting work. However, the epiphysis plates used in epiphysis part are often not uniform in width. As a result, many burrs occur in a narrow portion of round bar material, resulting in low yield. To reduce burr generation in the narrow portion, it is necessary to change the diameters of the round bar corresponding to a wide portion and the narrow portion. This requires cutting work of the round bars, which makes it difficult to improve the yield.

### PRIOR ART

### Non-Patent Literatures

Non-Patent Literature 1: Fiscal year 2018

### Strategic Foundation Technology Advancement and Collaboration Support Project

### Strategic Foundation Technology Advancement Support Project

“Development of high-quality, low-cost molding and processing technology for epiphysis plates”
Research and development results report
May 2019, responsible bureau: Kanto Bureau of Economy, Trade and Industry
Subsidiary business operator: Fujinokuni Medical Castle Town Promotion Organization https://www.chusho.meti.go.jp/keiei/sapoin/portal/seika/2016/2831803029h.pdf

### SUMMARY OF THE INVENTION

### Problems to Be Solved By the Invention

On the other hand, forward extrusion forging is known as a method for changing the diameter of a round bar in the wide and narrow portions of an epiphysis plate without cutting work of the round bar. However, in forward extrusion forging, there is a problem in that it is difficult to achieve the equiaxed microstructure required by ISO 20160 because the crystal grains elongate in the extrusion direction.

Therefore, the present invention proposes a manufacturing method for an epiphysis plate that uses forward extrusion forging and can achieve the equiaxed structure required by ISO 20160.

### Means for Solving the Problems

According to the present invention, the above problems are solved as follows.
(1) A manufacturing method of an epiphysis plate, the method including:
   a step of heating a metal material of a round bar;
   a step of forming a billet having a head portion with a large diameter and a shaft portion
with a small diameter integral with the head portion by a forward extrusion device;
   a step of heating the billet;
   a step of forming a die forged product close to a shape of the epiphysis plate as a final product by die forging using a die device, and
   a step of annealing the die forged product.

By using the manufacturing method of an epiphysis plate according to the present invention, the billet in which crystal grains are elongated in the extrusion direction by the forward extrusion device is die forged using the die device, resulting in the die forged product in which the strain of the elongated crystal grains has increased. By annealing this die forged product, the above-mentioned strain can be released and the crystal grains can be refined

Generally, annealing can relieve internal strain and restore coarsened crystal grains to a standard microstructure. However, in the present invention, the strain caused by elongating the crystal grains by forward extrusion is further increased by die forging. By performing annealing in this state, the crystal grains are more effectively refined. In this way, the equiaxed microstructure required by ISO 20160 can be achieved.
(2) In the above item (1), the billet is subjected to die forging using the die device one time or two or more times.

The step can be simplified by manufacturing a product of a desired shape in a single die forging. Furthermore, by dividing the die forging into several times, products with complex shapes can be manufactured by die forging.
(3) In the above item (1) or (2), the head portion of the billet corresponds to a wide portion of the epiphysis plate as the product, and the shaft portion of the billet corresponds to a narrow portion of the epiphysis plate.

By making the shape of the billet correspond to the shape of the epiphysis plate as a product, the material yield can be improved.
(4) In the above item (1) or (2), the metal material of the round bar is pure titanium or titanium alloy.
(5) In the above item (1) or (2), the metal material of the round bar is cobalt-chromium-molybdenum alloy.
(6) In the above item (1) or (2), the metal material of the round bar is austenitic stainless steel.

### BRIEF DESCRIPTION OF THE DRAWINGS

FIG. 1 shows a process diagram of a manufacturing method for an epiphysis plate of the present invention.

FIG. 2 shows a schematic diagram of a forward extrusion device used for the forward extrusion in FIG. 1.

FIG. 3 shows a front view of the die device used in die forging of FIG. 1.

FIG. 4A shows photographs A1-A6 of the equiaxed microstructure required by ISO 20160.

FIG. 4B shows photographs A7-A12 of the equiaxed microstructure required by ISO 20160.

FIG. 5 shows a photograph of the metal structure after forward extrusion, after die forging, and after annealing.

### EMBODIMENTS OF THE INVENTION

The following is a description of the embodiment of the present invention with reference to the drawings. In each drawing, similar parts are marked with the same reference sign.

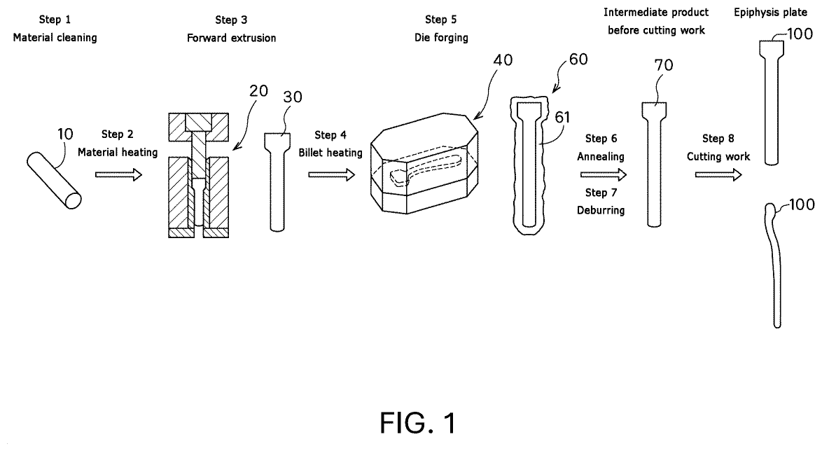

FIG. 1 shows a process diagram of a manufacturing method for an epiphysis plate 100 of the present invention. This process is composed of step 1: material cleaning, step 2: material heating, step 3: forward extrusion, step 4: billet heating, step 5: die forging, step 6: annealing, step 7: deburring, and step 8: cutting work.

In the manufacturing method of the epiphysis plate 100 of the present invention, the material 10 used in step 1 is a metal round bar, specifically, a round bar of Ti-6Al-4V ELI titanium alloy, cobalt-chromium-molybdenum alloy, austenitic stainless steel, or the like. In step 1, impurities and grease adhering to the material 10 are removed. Specifically, since mineral oil-based greases are the target, semi-aqueous detergents that combine water with a petroleum solvent containing a surfactant are generally used.

In the material heating of step 2, for example, a round bar of the material 10 made of Ti-6Al-4V ELI titanium alloy is heated to a temperature of the β transformation point or below. It is preferable to allow sufficient time to reach a uniform temperature to the center of the round bar.

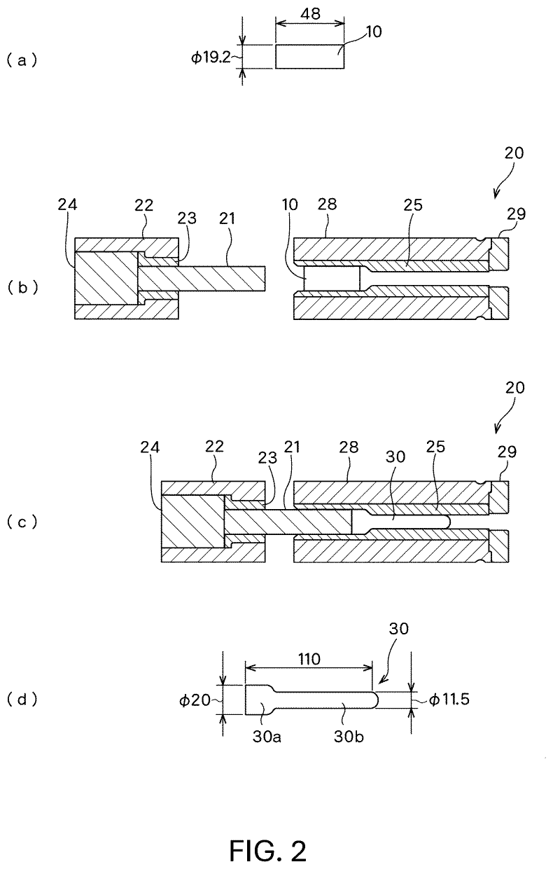

The forward extrusion in step 3 is a step in which a round bar (material) 10 heated to a temperature of the β transformation point or below is loaded into a cylindrical die 25 (see FIG. 2) using a forward extrusion device 20, and extruded forward with a punch 21 (see FIG. 2) to produce a billet 30 having a shape suitable for die forging. Step 3 will be further explained using FIG. 2.

FIG. 2(a) shows a round bar as a material 10, FIG. 2(b) and 2(c) show a forward extrusion device 20, and FIG. 2(d) shows a billet 30 produced by forward extrusion. The size of the round bar as the material 10 is, for example, 19.2 mm in outer diameter and 48 mm in length. When the material shown in FIG. 2(a) is used, the billet 30 produced by forward extrusion has a head portion with a larger diameter having an outer diameter of 20 mm, a shaft portion with a smaller diameter having an outer diameter of 11.5 mm, and a total length of 110 mm.

FIG. 2(b) is a schematic diagram of the forward extrusion device 20 loaded with material 10. By applying a “forward” pushing force to the punch 21 through the shim 24, the loaded material 10 is formed to match the shape of the die 25. In this forward extrusion device 20, a punch 21 is held by a punch guide 23 incorporated in a punch case 22. The die 25 is built into a die case 28, the lower part of which is blocked by a packing block 29.

FIG. 2(c) shows the forward extrusion device with the material 10 being extruded forward by the punch 21 to form the billet 30. By adopting forward extrusion in this manner, it is possible to mold a billet 30 in which the head portion 30a having a large diameter and the shaft portion 30b having a small diameter are integrally formed, thereby reducing the loss of material due to cutting work.

FIG. 2(d) shows a billet 30 formed by the forward extrusion device. In step 4 of FIG. 1, this billet 30 is heated to a temperature of the β transformation point or below as in step 2, and then used for die forging using die device 40 in step 5.

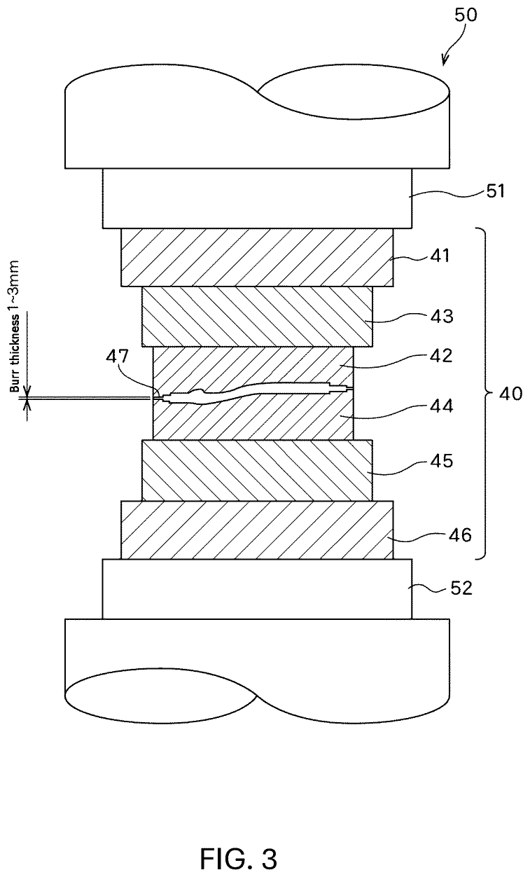

FIG. 3 shows a front view of the die device 40 used in die forging in step 5 of FIG. 1. The die device 40 are arranged in pairs, upper and lower, and each is attached to a press machine 50. The billet 30 is placed between the upper and lower pairs of the die device 40, and the pressing force of the press machine 50 deforms the billet 30 into the shape of the die device 40.

In FIG. 3, a die set upper mold 41 of the die device 40 is attached to a slide 51 of the press machine 50, and the die set upper mold 41 moves up and down together with a punch plate 43 and a punch 42 as the press machine 50 moves up and down. On the other hand, a die set lower mold 46 of the die device 40 is fixed to a bolster 52 of a press machine 50, and the die set lower mold 46, together with a die plate 45 and a die 44, receives the pressing force of the press machine 50 via the punch 42.

In addition, the die device 40 has a burr escape portion 47 formed between upper and lower pairs of die device 40 in order to escape burrs that are generated when the billet 30 is deformed. Although the gap of this burr escape portion 47 differs depending on the degree to which the billet 30 is deformed, it is usually adjusted to be within the range of 1 to 3 mm. As shown in step 5 of FIG. 1, a die forged product 60 formed by the die forging has a burr 61 formed over an entire periphery.

In the present invention, annealing is performed in step 6, and deburring is performed in step 7. In the case of Ti-6Al-4V ELI titanium alloys, annealing can restore the uniform α-β two-phase metal structure that was present in material 10. Furthermore, the hardness of the metal structure increased by die forging can be reduced. Therefore, the tool life can be improved in deburring in step 7, where burrs 61, which are unwanted protrusions, are removed by machining. The annealing is performed at 700 to 750 °C for 1 to 4 hours. By deburring in step 7, the die forged product 60 becomes an intermediate product before cutting work 70.

After deburring in step 6, annealing may be performed in step 7.

The cutting work of step 8, which is the final step, is performed on the intermediate product before cutting work 70. The cutting work involves two machining operations: surface machining and hole machining. The surface machining is an operation for removing scale formed on the surface of the intermediate product before cutting work 70 by forward extrusion and die forging, and for machining the product to a target dimension. The hole machining is an operation of cutting screw holes for inserting fixing screws that fasten the epiphysis plate 100 as a product to the bone.

A machining center is used for cutting work in step 8. The machining center is an NC milling machine with an automatic tool changing function. A number of cutting tools are stored in a tool magazine. The machining center automatically changes tools by computer numerical control commands to perform various machining operations such as milling, boring, drilling, and threading in succession. The epiphysis plate 100 is completed as a final product by cutting work

Note that surface treatment (sterilization treatment) may be carried out after cutting work.

The manufacturing method of the epiphysis plate 100 of the present invention employs forward extrusion forging, which does not require cutting work, in the step of producing a billet from a round bar of the material 10. This method takes advantage of the disadvantage of forward extrusion forging, in which the crystal grains are elongated in the extrusion direction. That is, the die forging after the forward extrusion forging increases the strain caused by the forward extrusion forging. By performing annealing in this state, it is possible to more effectively refine the crystal grains. By using the manufacturing method of the epiphysis plate 100 of the present invention in this manner, the yield of the material 10 can be further improved. In addition, the equiaxed microstructure required by ISO 20160 can be achieved.

In the process diagram in FIG. 1, the die forging in step 5 is performed once, but the die forging may be performed two or more times. In this case, the billet heating in step 4 and the die forging in step 5 are repeated two or more times. Although it is possible to form the intermediate product, which is the intermediate product before cutting work 70, by a single die forging, by performing multiple die forging, products with complex shapes can be produced by die forging.

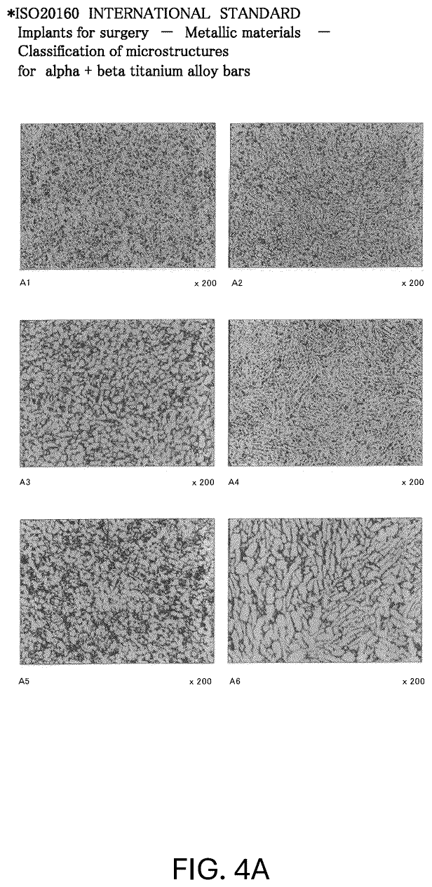

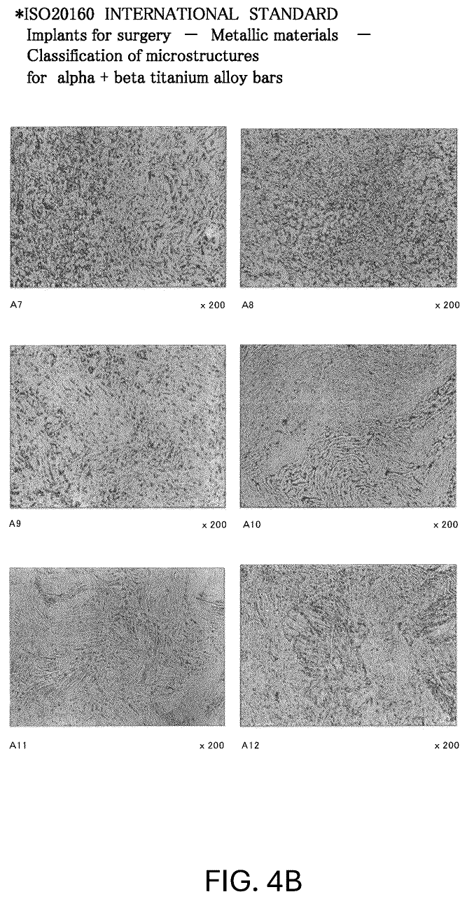

FIGS. 4A and 4B show photographs of the microstructure of ISO 20160 International Standard Implants for surgery-Metallic materials-alpha+beta titanium alloy bars. For example, in the photograph of A2 in FIG. 4A, a two-layer structure of α+β type constitutes an equiaxed microstructure.

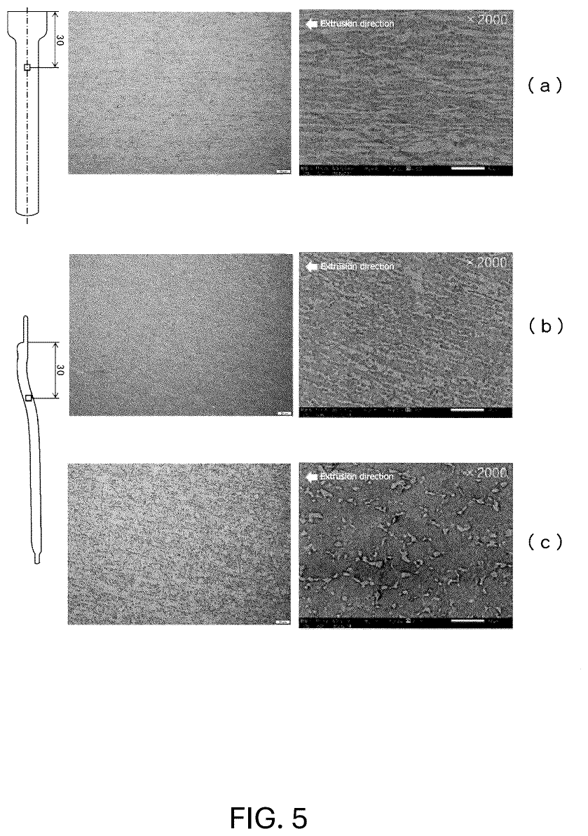

FIG. 5 is a photograph of a microstructure after processing in each step in the manufacturing method of the epiphysis plate 100 of the present invention. This photograph shows the microstructure of the center portion of a vertical section in the elongation direction of material 10, as shown on the left side of FIG. 5. FIG. 5(a) is a photograph of the metal structure after forward extrusion, FIG. 5(b) after die forging, and FIG. 5(c) after annealing.

FIG. 5(a) shows that the microstructure is elongated in the extrusion direction by forward extrusion. The photograph on the right side of FIG. 5(a) shows the microstructure at a magnification of 2000 times, and the photograph on the left side shows the microstructure at a magnification of 200 times. This 200× magnification is the same magnification as the ISO 20160 international standard photographs of FIGS. 4A and 4B. FIG. 5(b) shows that the strain in the microstructure, which is elongated in the extrusion direction, is increased by die forging. FIG. 5(c) clearly shows that the annealing process removes the increased microstructural strain, refines the grains, and forms an equiaxed microstructure. The photograph on the left side of FIG. 5(c) shows a microstructure equivalent to the equiaxed microstructure of A1-A2 in FIG. 4A.

In this embodiment, the manufacturing method of the epiphysis plate 100 using Ti-6Al-4V ELI titanium alloy as the main material has been described. However, the manufacturing method of the present invention for the epiphysis plate, i.e., the method of performing die forging after forward extrusion, can be applied to other materials that can be used as the epiphysis plate other than Ti-6Al-4V ELI titanium alloy. For example, a similar manufacturing method can be applied to pure titanium (CP-Ti), titanium alloys Ti-6Al-4V, Ti-6Al-2Nb-1Ta, Ti-15Zr-4Nb-4Ta, Ti-6Al-7Nb, Ti-3Al-2.5V, Ti-13Nb-13Zr, cobalt-chromium-molybdenum alloys, cobalt-chromium-tungsten-nickel alloys, austenitic stainless steel, and austenitic low-nickel stainless steel.

### REFERENCE SIGNS LIST

10 material
20 forward extrusion device
21 punch
22 punch case
23 punch guide
24 shim
25 die
28 die case
29 packing block
30 billet
30a head portion
30b shaft portion
40 die device
41 die set upper mold
42 punch
43 punch plate
44 die
45 die plate
46 die set lower mold
47 burr escape portion
50 press machine
51 slide
52 bolster
60 die forged product
61 burr
70 intermediate product before cutting work
100 epiphysis plate

What is claimed is:

1. A manufacturing method of an epiphysis plate, the method including:
a step of heating a metal material of a round bar;
a step of forming a billet having a head portion with a large diameter and a shaft portion
with a small diameter integral with the head portion by a forward extrusion device;
a step of heating the billet;
a step of forming a die forged product close to a shape of the epiphysis plate as a final product by die forging using a die device, and
a step of annealing the die forged product.

2. The manufacturing method of the epiphysis plate according to claim 1, wherein
the billet is die forged using the die device one time or two or more times.

3. The manufacturing method of the epiphysis plate according to claim 1 or 2, wherein the head portion of the billet corresponds to a wide portion of the epiphysis plate as the final product, and the shaft portion of the billet corresponds to a narrow portion of the epiphysis plate.

4. The manufacturing method of the epiphysis plate according to claim 1 or 2, wherein the metal material of the round bar is pure titanium or titanium alloy.

5. The manufacturing method of the epiphysis plate according to claim 1 or 2, wherein the metal material of the round bar is cobalt-chromium-molybdenum alloy.

6. The manufacturing method of the epiphysis plate according to claim 1 or 2, wherein the metal material of the round bar is austenitic stainless steel.
